(19) United States
(12) Patent Application Publication
(10) Pub. No.: US 2011/0117038 A1
(43) Pub. Date: May 19, 2011

(54) Dopa Oxidase Inhibitor, Skin-Lightening Agent and External Preparation for Skin

(75) Inventors: Akiko KAWASAKI, Haga-gun (JP); Mitsuru Sugiyama, Haga-gun (JP); Yasuko Amano, Tokyo (JP)

(73) Assignee: Kao Corporation, Tokyo (JP)

(21) Appl. No.: 12/939,349

(22) Filed: Nov. 4, 2010

(30) Foreign Application Priority Data
Nov. 19, 2009 (JP) 2009-263588

(51) Int. Cl. A61K 8/97; A61K 8/49; A61K 36/9064; A61K 36/28; A61K 36/88; A61K 31/58; C07J 71/00; A61Q 19/02; A61Q 19/00; A61P 17/00

(52) U.S. Cl. 424/62; 424/756; 424/725; 514/172; 540/83

(57) ABSTRACT

A dopa oxidase inhibitor, a skin-lightening agent and an external preparation for skin; comprising an extract of at least one plant selected from the group consisting of Melia toosendan Sieb. et Zucc., Amomum tsao-ka Crevost et Lemaire. Senecio gracilis and Veratrum nigrum L., or a compound represented by the following Formula (1), as an active ingredient:

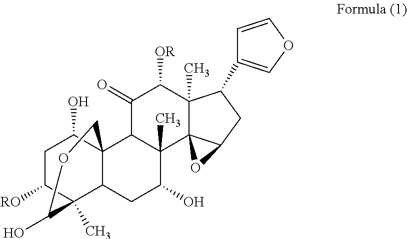

wherein R represents an acyl group having 1 to 5 carbon atom(s).

### FIELD OF THE INVENTION

The present invention relates to a dopa oxidase inhibitor, a skin-lightening agent, and an external preparation for skin.

### BACKGROUND OF THE INVENTION

Pigmentation after suntan, and blotches and freckles are generally considered to be generated as a result of an activation of pigment cells (melanocytes) presented in a skin followed by an increase in a melanin production, due to a stimulation of the skin by an ultraviolet exposure, abnormal secretion of hormones, genetic factors, or the like. In an organism, a pigment melanin is biosynthesized from tyrosine, which is a precursor of the melanin, at the melanosome in the pigment cells (melanocytes) under an action of an enzyme tyrosinase. It has been reported that when a mutation(s) is occurred in the tyrosinase, which is an enzyme involved in a melanin biosynthesis, a formation of a melanin pigment in a skin and a hair becomes anomalous (King R A. Oetting W S. Hearing V J. in Metabolic bases of inherited disease (Scriver C R. Beaudet A L. Sly W S. Valle D., eds.), McGraw-Hill, New York, 4353-4392, 1995).

Due to the significance of tyrosinase in the melanin biosynthesis, tyrosinase has been paid attention as a target of skin lightening materials since early times. Tyrosinase is the enzyme, specifically, having a tyrosine hydroxylase activity, a dopa oxidase activity, and a DHI activity, and catalyzes a reation of a melanin synthesis which uses tyrosine and dopa as precursors. Therefore, when investigating the tyrosinase enzymatic activity, for example, the dopa oxidase activity or the like can be used as an index. In fact, the dopa oxidase activity has been used as an index, when evaluating a suppressing material(s) of a melanin production which has an inhibitory effect of a tyrosinase enzymatic activity (Wrathall J R. et al., JCB 1973 57: 406-423). According to this technique, when the dopa oxidase activity in the melanocyte is suppressed, the production of melanin as the final biosynthesis product can be suppressed.

As such, a research has been hitherto made on substances capable of inhibiting an activity of tyrosinase and thereby suppressing melanin production, reducing melanin that has been produced, and the like, and use of these substances as skin-lightening components has been investigated. For example, ascorbic acid, arbutin, kojic acid, glutathione and the like have been reported to have a relevant action (for example, Skin-Lightening Strategy (Nankodo Co., Ltd.) IV. Pharmacology and Clinical Practice for Skin-Lightening Agents, p 95-115).

However, these substances may also have problems such as the effect of suppressing melanin production being insufficient, and thus a sufficiently satisfactory substance for the purpose has not yet been obtained.

### SUMMARY OF THE INVENTION

It is an object of the present invention to provide a dopa oxidase inhibitor which is capable of effectively inhibiting a dopa oxidase activity. It is another object of the present invention to provide a skin-lightening agent and an external preparation for skin, both of which suppress melanin production by inhibiting the dopa oxidase activity.

The present invention resides in a dopa oxidase inhibitor, a skin-lightening agent and an external preparation for skin, comprising an extract of at least one plant selected from the group consisting of Melia toosendan Sieb. et Zucc., Amomum tsao-ka Crevost et Lemaire, Senecio gracilis and Veratrum nigrum L. as an active ingredient.

Further, the present invention resides in a dopa oxidase inhibitor, a skin-lightening agent, comprising a compound represented by the following Formula (1) as an active ingredient:

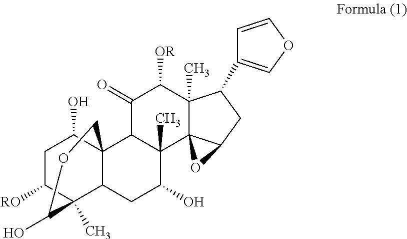

wherein R represents an acyl group having 1 to 5 carbon atom(s).

Further, the present invention resides in a method for inhibiting a dopa oxidase activity and a method for lightening a skin, comprising applying an extract of at least one plant selected from the group consisting of Melia toosendan Sieb. et Zucc., Amomum tsao-ka Crevost et Lemaire, Senecio gracilis and Veratrum nigrum L. to a skin.

Further, the present invention resides in a method for inhibiting a dopa oxidase activity and a method for lightening a skin, comprising applying the compound represented by the above Formula (1) to a skin.

### DETAILED DESCRIPTION OF THE INVENTION

The inventors of the present invention found that an extract of a certain kind of plant has an inhibitory action of a dopa oxidase activity. The inventors of the present invention also found that an excellent dopa oxidase inhibitor, skin-lightening agent and external preparation for skin can be provided by using the extract.

Furthermore, the inventors of the present invention found that a compound represented by the following Formula (1) has an inhibitory action on the dopa oxidase activity, and obtained a finding that this compound is useful as a novel skin-lightening component.

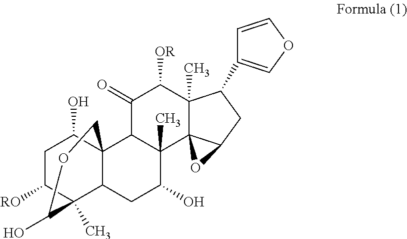

wherein R represents an acyl group having 1 to 5 carbon atom(s).

According to the present invention, there is provided the following means:

(1) A dopa oxidase inhibitor, comprising an extract of at least one plant selected from the group consisting of Melia toosendan Sieb. et Zucc., Amomum tsao-ka Crevost et Lemaire. Senecio gracilis and Veratrum nigrum L. as an active ingredient.
(2) A skin-lightening agent, comprising an extract of at least one plant selected from the group consisting of Melia toosendan Sieb. et Zucc., Amomum tsao-ka Crevost et Lemaire. Senecio gracilis and Veratrum nigrum L. as an active ingredient.
(3) An external preparation for skin, comprising an extract of at least one plant selected from the group consisting of Melia toosendan Sieb. et Zucc., Amomum tsao-ka Crevost et Lemaire. Senecio gracilis and Veratrum nigrum L. as an active ingredient.
(4) A dopa oxidase inhibitor, comprising the compound represented by the above Formula (1) as an active ingredient.
(5) A skin-lightening agent, comprising the compound represented by the above Formula (1) as an active ingredient.
(6) A method for inhibiting a dopa oxidase activity, comprising applying an extract of at least one plant selected from the group consisting of Melia toosendan Sieb. et Zucc., Amomum tsao-ka Crevost et Lemaire. Senecio gracilis and Veratrum nigrum L. to a skin.
(7) A method for lightening a skin, comprising applying an extract of at least one plant selected from the group consisting of Melia toosendan Sieb. et Zucc., Amomum tsao-ka Crevost et Lemaire, Senecio gracilis and Veratrum nigrum L. to a skin.
(8) A method for inhibiting a dopa oxidase activity, comprising applying the compound represented by the above Formula (1) to a skin.
(9) A method for lightening a skin, comprising applying the compound represented by the above Formula (1) to a skin.

According to the present invention, the term “skin-lightening (action)” means an action of suppressing the production of melanin pigment and thereby returning a skin color to the original transparent tone without any extra melanin, or an action of preventing and/or suppressing darkening of a skin or pigmentation such as blotches and freckles.

The dopa oxidase inhibitor of the present invention can effectively inhibit the dopa oxidase activity. Furthermore, the skin-lightening agent and external preparation for skin of the present invention can suppress the tyrosinase-induced melanin production by suppressing the dopa oxidase activity, to thereby show prophylactic or preventive effects on blotches and freckles or on pigmentation after suntan.

Other and further features and advantages of the present invention will appear more fully from the following description.

The present invention is described more detail below.

Each of the dopa oxidase inhibitor, the skin-lightening agent and the external preparation for skin of the present invention contains an extract of at least one plant selected from the group consisting of Melia toosendan Sieb. et Zucc., Amomum tsao-ka Crevost et Lemaire. Senecio gracilis and Veratrum nigrum L.; or the compound represented by the above Formula (1); as an active ingredient.

First, the dopa oxidase inhibitor, the skin-lightening agent and the external preparation for skin of the present invention, all of which contain an extract of a certain plant(s) (hereinafter, “plant extract”) as an active ingredient, are explained below.

The plant extract has an inhibitory effect on a dopa oxidase activity. Therefore, by containing the plant extract, the skin-lightening agent and external preparation for skin of the present invention can suppress melanin production in the skin and show a skin-lightening effect.

Melia toosendan Sieb. et Zucc. according to the present invention is a plant belonging to the Meliaceae.

Amomum tsao-ka Crevost et Lemaire according to the present invention is a plant belonging to the Zingiberaceae.

Senecio gracilis according to the present invention is a plant belonging to the Asteraceae.

Veratrum nigrum L. according to the present invention is a plant belonging to the Liliaceae.

According to the present invention, any and all parts of the plant can be used. For example, any one or more selected from the whole tree of the plant, or any part (roots, rhizomes, trunks, branches, stems, leaves, barks, tree sap, tree resin, flowers, fruits, seeds, and the like), and combinations of those, can be used.

According to the present invention, in order to obtain an extract of Melia toosendan Sieb. et Zucc., it is preferable to use the fruits of the plant, and an herbal medicine (Senrenshi) obtained by using Melia toosendan Sieb. et Zucc. as the original plant can also be used.

According to the present invention, in order to obtain an extract of Amomum tsao-ko Crevost et Lemaire, it is preferable to use the fruits of the plant, and an herbal medicine (Souka) obtained by using Amomum tsao-ka Crevost et Lemaire as the original plant can also be used.

According to the present invention, in order to obtain an extract of Senecio gracilis, it is preferable to use the roots of the plant.

According to the present invention, in order to obtain an extract of Veratrum nigrum L. it is preferable to use the rhizomes of the plant, and an herbal medicine (Kokuriro, Riro, Reiro) obtained by using Veratrum nigrum L. as the original plant can also be used.

The plant extract used in the present invention can be prepared according to a conventional method of extraction using an appropriate solvent.

According to the present invention, the plant can be used directly or after drying and grinding for the preparation of the plant extract. A steam distillate or a compression product thereof can also be used for the preparation of the plant extract. These can also be used in a further purified form, such as essential oil. In addition, commercially available products can also be used. The plant, a steam distillate thereof and compression product thereof may be used singly, or in combination of two or more kinds.

As the solvent used for extraction, those typically used for the extraction of plant components are usable. Examples thereof include water, petroleum ether, n-hexane, toluene, chloroform, ether, ethyl acetate, acetone, methanol, ethanol, propanol, butanol, ethylene glycol, propylene glycol, and butylene glycol. Particularly, water, ethanol, propylene glycol and butylene glycol are preferable. These can be used singly, or in combination of two or more kinds. With regard to the extraction condition, typical extraction conditions can be employed. For example, the above-described plant may be dipped or heated under reflux for two hours to 60 days at 5 to 80° C. The plant extract can be directly used, but may also be used as a fraction with high activity obtained by fractionation using appropriate separating techniques, for example, gel filtration, chromatography, precision distillation and activated carbon treatment.

According to the present invention, the plant extract may be used directly. Further, the plant extract may be diluted, concentrated or freeze-dried, and then prepared into a powder or a paste before use.

The plant extract described above shows an inhibitory action of the dopa oxidase activity. When such a plant extract is incorporated as an active ingredient, the dopa oxidase inhibitor, the skin-lightening agent and the external preparation for skin of the present invention can be obtained.

According to the present invention, the plant extract may be directly used as a dopa oxidase activity inhibitor, a skin-lightening agent and an external preparation for skin. Alternatively, the extract may also be used as a formulation prepared by adding thereto an appropriate liquid or solid excipient or extending agent such as, for example, titanium oxide, calcium carbonate, distilled water, lactose or starch. In this case, the amount of the plant extract is not particularly limited, but it is preferable that the extract be contained in an amount of 0.00001 to 5% by mass, and particularly preferably 0.0001 to 0.5% by mass, in terms of the solids content.

Next, the dopa oxidase activity inhibitor and skin-lightening agent containing a component represented by the following Formula (1) as an active ingredient are explained below.

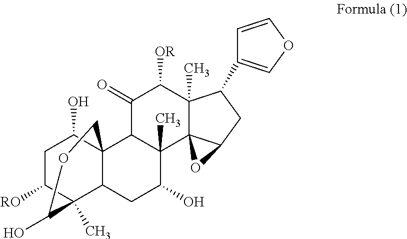

In Formula (1), R represents an acyl group having 1 to 5 carbon atom(s).

R is preferably an acyl group having 1 to 3 carbon atom(s), and more preferably an acyl group having two carbon atoms. Specific examples of R include a formyl group, an acetyl group, a propionyl group, a butyryl group, an isobutyryl group, a valeryl group, and an isovaleryl group. Among them, a formyl group, an acetyl group, and a propionyl group are preferred, and an acetyl group is more preferred. A compound represented by the Formula (1) in which R is an acetyl group is Toosendanin, which is a kind of terpene.

The compound represented by the Formula (1) may adopt tautomeric forms as shown below, and the compound represented by the above Formula (1) of the present invention includes both of the tautomers.

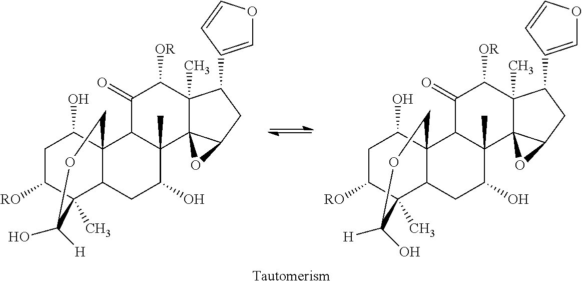

The method for producing the compound represented by the above Formula (1) used in the present invention is not particularly limited. A chemically synthesized compound may be used, and a compound extracted or purified from a natural product-derived material may also be used. Furthermore, a product that is commercially available as a reagent can also be used.

As the reagent, a product commercially available from Avachem Scientific LLC (USA) and the like can be used.

In regard to the method of obtaining the compound represented by the above Formula (1) from a natural product-derived material, for example, the compound can be isolated from plants such as Melia toosendan Sieb. et Zucc., Melia azedarach, and Toona sinensis.

For these plants, any and all parts (whole tree, whole herb, roots, rhizomes, trunks, branches, stems, leaves, barks, tree sap, tree resin, flowers, fruits, seeds and the like of the plant) can all be used. Particularly, in the case of isolating the compound from Melia toosendan Sieb. at Zucc., it is preferable to use the barks, seeds or fruits of the plant. Furthermore, an herbal medicine obtained by using the fruits of Melia toosendan Sieb. et Zucc. as the original plant, Senrenshi, can also be used. In the case of isolating the compound from Melia azedarach, it is preferable to use the barks of the plant. An herbal medicine obtained by using the barks of Melia azedarach as the original plant. Kurenpi, can also be used. In the case of isolating the compound from Toona sinensis, it is preferable to use the barks of the plant.

The compound represented by the Formula (1) can also be isolated by using these various plants or various parts in appropriate combination.

The method of isolating the compound represented by the above Formula (1) from these plants is not particularly limited, but an example may be a method of extracting the plant(s) mentioned above using an appropriate solvent, and isolating the compound represented by the above Formula (1) from the obtained plant extract by a technique such as chromatography.

The plant can be used directly or after drying and grinding for the preparation of the plant extract. As the solvent that is used for the extraction, those conventionally used for the extraction of plant components, for example, water, petroleum ether, n-hexane, toluene, chloroform, ether, ethyl acetate, acetone, methanol, ethanol, propanol, butanol, ethylene glycol, propylene glycol, butylene glycol and mixed solutions thereof, can be used. With regard to the extraction condition, typical extraction conditions can be employed. For example, the above-described plant may be dipped or heated under reflux for two hours to 60 days at 5 to 80° C.

Specific methods for extraction and isolation that can be used include the methods shown in the following Examples, but the present invention is not intended to be limited thereto.

As demonstrated in the Examples described below, the compound represented by the above Formula (1) effectively suppresses the dopa oxidase activity. Therefore, the skin-lightening agent of the present invention containing the compound can inhibit tyrosinase activity and shows a skin-lightening effect by suppressing the production of melanin in the skin. Furthermore, the compound represented by the above Formula (1) can also be used as a dopa oxidase inhibitor or a tyrosinase inhibitor.

JP-T-2006-514657 discloses that a composition containing Toosendanin is used in the prevention and improvement of wrinkles. It is also known that the herbal medicines. Senrenshi and Kurenpi, show an anthelmintic action. However, the fact that the compound represented by the above Formula (1) shows an action of effectively inhibiting dopa oxidase activity and is useful as a skin-lightening component is a finding newly obtained by the inventors of the present invention.

According to the present invention, the compound represented by the above Formula (1) may be directly used as a dopa oxidase inhibitor or a skin-lightening agent. Alternatively, the compound may also be used as a formulation prepared by adding thereto an appropriate liquid or solid excipient or extending agent such as titanium oxide, calcium carbonate, distilled water, lactose or starch. In this case, the amount of the compound represented by the above Formula (1) is not particularly limited, but it is preferable that the compound represented by the Formula (1) be contained in an amount of 0.00001 to 3% by mass, and more preferably 0.01 to 1% by mass.

Particularly, in the case of using the compound represented by the above Formula (1) as a skin-lightening agent, other skin-lightening component(s) may be used in addition to the compound. When the skin-lightening agent of the present invention is used as a composition together with other skin-lightening component(s), the amount of the compound represented by the above Formula (1) is not particularly limited, but it is preferable that the compound represented by the above Formula (1) be contained in an amount of 0.00001 to 3% by mass, and more preferably 0.01 to 1% by mass.

The dopa oxidase inhibitor and the skin-lightening agent of the present invention can be used in the form of an external preparation for skin. The “external preparation for skin” means a formulation that is applied to the skin as a cosmetic material for skin, a drug for external use, a quasi-drug for external use, or the like. The dosage form can be in a wide variety of forms such as an aqueous solution system, a solubilized system, an emulsified system, a powder system, a gel system, an ointment system, a cream, water-oil biphasic system, and a water-oil-powder triphasic system. Examples of the dosage form include a face wash, a skin toner, an emulsion, a cream, a gel, an essence (serum), a facial pack, a facial mask, a foundation, an ointment, and a sheet-like product.

When used in the form of an external preparation for skin, components that are used in conventional external preparations for skin, for example, a surfactant, an oily material, a polymer compound, a preservative, a skin aging preventing agent, efficacious ingredients other than the skin-lightening components mentioned above, a powder, an ultraviolet absorbent, a colorant, a fragrance, an emulsification stabilizer, and a pH adjusting agent can be appropriately incorporated into the external preparation, in addition to the above-mentioned plant extract or the compound represented by the above Formula (1). The dosage amount of the external preparation for skin containing the plant extract or the compound represented by the above Formula (1) may vary with the content of the active ingredient, however, for example, in the case of a cream form or an ointment form, the dosage amount is preferably 0.1 to 5 μg per square centimeter of a skin, and in the case of a liquid preparation, the dosage amount of use is preferably 0.1 to 10 μg per square centimeter of a skin.

The present invention will be described in more detail based on the following examples, but the invention is not intended to be limited thereto.

### EXAMPLES

### Preparation Example 1

### Preparation of Extract of Melia toosendan Sieb. et Zucc.

50 g of the herbal medicine Senrenshi (purchased from Shinwa Bussan Co., Ltd.), which uses Melia toosendan Sieb. et Zucc. as an original plant, was finely chopped, and 500 mL of 50% ethanol was added thereto. Extraction was carried out for 3 days at room temperature, and then the liquid was filtered to obtain a crude extract liquid (yield 343 mL, evaporation residue 3.08 w/v %). The crude extract liquid was diluted to be an evaporation residue of 1.0 w/v %, and thus an extract of Melia toosendan Sieb. et Zucc. was prepared.

### Preparation Example 2

### Preparation of Extract of Amomum tsao-ka Crevost et Lemaire

150 g of the herbal medicine Sooka (purchased from Shinwa Bussan Co., Ltd.), which uses Amomum tsao-ka Crevost et Lemaire as an original plant, was finely chopped, and 500 mL of 50% ethanol was added thereto. Extraction was carried out for 2 days at room temperature, and then the liquid was filtered to obtain a crude extract liquid (yield 248 mL, evaporation residue 2.54 w/v %). The crude extract liquid was diluted to be an evaporation residue of 1.0 w/v %, and thus an extract of Amomum tsao-ka Crevost et Lemaire was prepared.

### Preparation Example 3

### Preparation of Extract of Senecio gracilis

40 g of the roots of Senecio gracilis (purchased from American Botanicals Company) was finely chopped, and 400 mL of 50% ethanol was added thereto. Extraction was carried out for 22 days at room temperature, and then the liquid was filtered to obtain a crude extract liquid (yield 298 mL, evaporation residue 1.76 w/v %). The crude extract liquid was diluted to be an evaporation residue of 1.0 w/v %, and thus an extract of Senecio gracilis was prepared.

### Preparation Example 4

### Preparation of Extract of Veratrum nigrum L.

50 g of the herbal medicine Reiro (purchased by Shinwa Bussan Co., Ltd.), which uses Veratrum nigrum L. as an original plant, was finely chopped, and 500 mL of 50% ethanol was added thereto. Extraction was carried out for 2 days at room temperature, and then the liquid was filtered to obtain a crude extract liquid (yield 384 mL, evaporation residue 1.51 w/v %). The crude extract liquid was diluted to have an evaporation residue of 1.0 w/v %, and thus an extract of Veratrum nigrum L. was prepared.

### Test Example 1

### Measurement of Dopa Oxidase Activity

100 μL of human neonatal foreskin-derived melanocytes were inoculated on each well of a 96-well plate to be a cell density of 1×10⁴ cells/well. The medium used therein was Medium 254 to which HMGS (Human Melanocyte Growth Supplement) excluding PMA (all manufactured by Cascade Biologics Inc.) was added.

After culturing the cells for 24 hours, endothelin-1 (ET-1) which is a melanocyte activator, stem cell growth factor (SCF), α-melanocyte stimulating hormone (α-MSH), histamine and prostaglandin E₂ (PGE₂) were mixed and added to the each culture at a final concentration in medium of 10×10⁻⁷ mol/m³ in each.

Then, the plant extracts prepared in Preparation Examples 1 to 4 were added to the cell culture to obtain a final concentration of 0.10 volume %. The cells were cultured for 3 days at a final amount of medium of 200 μL/well under the conditions of 37° C., 5% CO₂.

The following additives had also been added to the medium.

| | | |
|---|---|---|
| bFGF (basic fibroblast growth factor) | 3 | ng/mL |
| BPE (Bovine Pituitary Extract) | 0.2 | volume % |
| FBS (Fetal bovine serum) | 0.5 | volume % |
| Hydrocortisone | 5 × 10⁻⁴ | mol/m³ |
| Insulin | 5 | μg/mL |
| Transferrin | 5 | μg/mL |
| Heparin | 5 | μg/mL |

After completion of the culture, 20 μL of Alamar Blue reagent (trade name, manufactured by Invitrogen Inc.) was added to each well, and the cells were incubated for 2 to 3 hours. Subsequently, the cell proliferation activity was measured by measuring the fluorescence intensity of the medium (excitation wavelength: 544 nm, fluorescence wavelength: 590 nm). The results are shown in Table 1.

The melanocytes which had been used in the measurement of the cell proliferation activity were washed with Phosphate-buffered saline (PBS) from which Ca²⁺ and Mg²⁺ had been eliminated, and 20 μL/well of an extraction buffer (0.1 M Tris-HCl (pH 7.2), 1% Nonidet P-40, 0.01% SDS, 100 μM phenylmethylsulfonyl fluoride (PMSF) and 1 μg/ml aprotinin) and 20 μL/well of an assay buffer (100 mM Sodium phosphate-buffer (pH 7.1) containing 4% dimethylformamide) were added to the cells. The cells were lysed for 3 hours at 4° C., and its dopa oxidase activity was measured. The measurement of the dopa oxidase activity was carried out as follows, with reference to the MBTH method (see, for example, Winder A. J., Harris H., Eur. J. Biochem., 198, 317-326, 1991).

To each well containing the lysed cell solution, 80 μL/well of the assay buffer, 60 μL of a 20.7 mM 3-methyl-2-benzothiazolinonehydrazone (MBTH) solution, and 40 μL of a 5 mM L-dopa (L-dihydroxyphenylalanine) solution were respectively added, and the mixture was allowed to react at 37° C. for 30 to 60 minutes. Subsequently, the color reaction was analyzed based on the absorbance at 490 nm.

The results are shown in Table 1. The values of the cell proliferation activity in Table 1 are indicated as values relative to the fluorescence intensities of the instances where various plant extracts were not added. Further, the values of the dopa oxidase activity are indicated as values relative to the absorbances of the instances where various plant extracts were not added.

TABLE 1

| | Cell proliferation | Dopa oxidase |
|---|---|---|
| Plant extract | activity (%) | activity (%) |
| Melia toosendan Sieb. | 108 | 44 |
| et Zucc. | | |
| Amomum tsao-ka | 128 | 60 |
| Crevost et Lemaire | | |
| Senecio gracilis | 131 | 80 |
| Veratrum nigrum L. | 104 | 83 |

As shown in Table 1, it was recognized that the extracts prepared in Preparation Examples 1 to 4 suppress the dopa oxidase activity. As described above, the dopa oxidase activity is used as an index of the enzyme activity of tyrosinases which is involved in melanin biosynthesis. Therefore, from the results of Table 1, it can be seen that the extracts prepared in Preparation Examples 1 to 4 suppress melanin production by inhibiting the dopa oxidase activity, and consequently show a skin-lightening action.

Further, it was recognized that the extracts prepared in Preparation Examples 1 to 4 cause an increase in the cell proliferation activity. Therefore, it was found that the plant extracts of the present invention do not have an effect of lowering the cell proliferation activity (namely, an effect of lowering the cell proliferation ability).

From the results obtained in Test Example 1, it was understood that the plant extracts of the present invention can inhibit the dopa oxidase activity without inhibiting cell proliferation activity.

### Preparation Example 5

### Isolation of Inhibitory Component of Dopa Oxidase Activity

800 g of the fruits of Melia toosendan Sieb. et Zucc. (Senrenshi, manufactured by Shinwa Bussan Co., Ltd.) was extracted with 8 L of 50% ethanol at 20 to 35° C. for 7 days, and the solvent was concentrated. Thus, 123.5 g of an extracted solid fraction was obtained. The obtained extracted solid fraction was fractionated based on the inhibitory activity of the dopa oxidase activity as an index. Liquid-liquid distribution was carried out using water and ethyl acetate, and the inhibitory activity was concentrated in 20.78 g of the ethyl acetate layer (yield 16.8%). The ethyl acetate layer was further fractionated by silica gel column chromatography, and thus Fraction (1) 7.49 g was obtained (yield 6.1%). Fraction (1) was further fractionated using silica gel column chromatography, and thus Fraction (2) 2.78 g was obtained (yield 2.3%). Subsequently, this fraction was fractionated using LH-20 (Sephadex (trade mark) LH20, manufactured by GE Healthcare Inc.), and thus Fraction (3) 1.28 g was obtained (yield 1.0%). 340 mg of Fraction (3) was fractionated by HPLC, and thus Fraction (4) 164.8 mg was obtained (yield 0.48%). Furthermore, 20 mg of Fraction (4) was fractionated by HPLC, and thus Fraction (5) 1.4 mg was obtained (yield 0.03%). Fraction (5) was subjected to a structure analysis using NMR. In the structure analysis based on NMR, toosendanin which is commercially available as a reagent (manufactured Avachem Scientific LLC) was used for comparison. The results of the structure analysis based on NMR are presented in Table 1.

As a result, the active component isolated from Melia toosendan Sieb. et Zucc. was a compound having a structure shown in the following Table 2, and this compound was identified as toosendanin. In Table 2, the abbreviation Ac represents an acetyl group.

TABLE 2

| | 13C-NMR (ppm) | | 1H-NMR (ppm) | |
|---|---|---|---|---|
| | Isolated active | Toosendanin | Isolated active | Toosendanin |
| | component | (reagent) | component | (reagent) |
| 1 | 70.7 | 70.7 | 4.24 | 4.24 |
| 2 | 37.2 | 37.2 | 1.8 | 1.8 |
| | | | 2.73 | 2.73 |
| 3 | 74.8 | 74.8 | 5.19 | 5.19 |
| 4 | 41.2 | 41.2 | — | — |
| 5 | 29.6 | 29.6 | 2.8 | 2.8 |
| 6 | 26.2 | 26.2 | 1.71 | 1.71 |
| | | | 2 | 2 |
| 7 | 70.8 | 70.9 | 3.57 | 3.57 |
| 8 | 43.9 | 43.9 | — | — |
| 9 | 50.1 | 50.1 | 4.7 | 4.7 |
| 10 | 42.9 | 42.9 | — | — |
| 11 | 209.1 | 209.1 | — | — |
| 12 | 79.7 | 79.7 | 5.33 | 5.33 |
| 13 | 46.9 | 46.9 | — | — |
| 14 | 73.6 | 73.6 | — | — |
| 15 | 60 | 60 | 3.81 | 3.8 |
| 16 | 34.8 | 34.8 | 2.02 | 2.01 |
| | | | 2.11 | 2.11 |
| 17 | 39.9 | 39.9 | 2.88 | 2.88 |
| 18 | 15.9 | 15.8 | 1.37 | 1.38 |
| 19 | 65.5 | 65.5 | 4.25 | 4.24 |
| | | | 4.31 | 4.31 |
| 20 | 124.3 | 124.3 | — | — |
| 21 | 142.1 | 142.1 | 7.2 | 7.2 |
| 22 | 113 | 113 | 6.16 | 6.16 |
| 23 | 143.7 | 143.7 | 7.4 | 7.4 |
| 28 | 20 | 20 | 0.84 | 0.84 |
| 29 | 97.2 | 97.2 | 4.83 | 4.83 |
| 30 | 23.1 | 23.2 | 1.12 | 1.12 |
| AcO | 21.4 | 21.4 | 2.07 | 2.07 |
| | 172.9 | 172.9 | — | — |
| | 20.9 | 20.9 | 1.95 | 1.95 |
| | 172.2 | 172.2 | — | — |

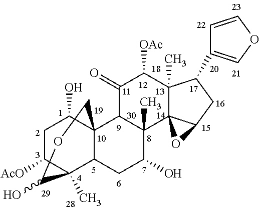

### Test Example 2

### Measurement of Dopa Oxidase Activity

100 μL of human neonatal foreskin-derived melanocytes were inoculated on each well of a 96-well plate to be a cell density of 1×10⁴ cells/well. The medium used therein was Medium 254 to which Human Melanocyte Growth Supplement excluding PMA (all manufactured by Cascade Biologics Inc.) was added.

After culturing the cells for 24 hours, endothelin-1 (ET-1) which is a melanocyte activator, stem cell growth factor (SCF), α-melanocyte stimulating hormone (α-MSH), histamine and prostaglandin E₂ (PGE₂) were mixed and added to the culture to obtain a final concentration in medium of 10×10⁻⁷ mol/m³ in each.

As a sample, toosendanin which is the compound isolated in Preparation Example 5 was added to the culture at a final concentration of 100 nM. Furthermore, samples for reference were produced by adding kojic acid to the culture at final concentrations of 10 μM and 1 mM. Kojic acid is a known skin-lightening component having high tyrosinase inhibitory activity.

The cells were cultured for 3 days at a final amount of medium of 200 μL/well under the conditions of 37° C. and 5% CO₂.

The following additives had also been added to the medium.

| | | |
|---|---|---|
| bFGF (basic fibroblast growth factor) | 3 | ng/mL |
| BPE (Bovine Pituitary Extract) | 0.2 | volume % |
| FBS (Fetal bovine serum) | 0.5 | volume % |
| Hydrocortisone | 5 × 10⁻⁴ | mol/m³ |
| Insulin | 5 | μg/mL |
| Transferrin | 5 | μg/mL |
| Heparin | 5 | μg/mL |

After completing culture, the melanocytes were washed with Phosphate-buffered saline (PBS) from which Ca²⁺ and Mg²⁺ had been eliminated, and 20 μL/well of an extraction buffer (0.1 M Tris-HCl (pH 7.2), 1% Nonidet P-40, 0.01% SDS, 100 μM phenylmethylsulfonyl fluoride (PMSF) and 1 μg/ml aprotinin) and 20 μL/well of an assay buffer (100 mM Sodium phosphate-buffer (pH 7.1) containing 4% dimethylformamide) were added to the cells. The cells were lysed for 3 hours at 4° C., and the dopa oxidase activity was measured. The measurement of the dopa oxidase activity was carried out as follows, with reference to the MBTH method (see, for example, Winder A. J., Harris H., Eur. J. Biochem., 198, 317-326, 1991).

To each well containing the lysed cell solution, 80 μL/well of the assay buffer, 60 μL of a 20.7 mM 3-methyl-2-benzothiazolinonehydrazone (MBTH) solution, and 40 μL of a 5 mM L-dopa (L-dihydroxyphenylalanine) solution were respectively added, and the mixture was allowed to react at 37° C. for 30 to 60 minutes. Subsequently, the color reaction was analyzed based on the absorbance at 490 nm.

The results are presented in Table 3. The values of the cell proliferation activity in Table 3 are indicated as values relative to the fluorescence intensities of the instances where sample were not added.

TABLE 3

| | | | Dopa oxidase | |
|---|---|---|---|---|
| Compound | Concentration | | activity (%) | Remark |
| Toosendanin | 100 | nM | 15 | Present invention |
| Kojic acid | 10 | μM | No inhibitory | Reference example |
| | | | activity | |
| Kojic acid | 1 | mM | 55 | Reference example |

As it is obvious from the results of Table 3, it was understood that in the system incorporated with the toosendanin isolated from Melia toosendan Sieb. et Zucc., the dopa oxidase activity was decreased to a large extent, and the dopa oxidase activity can be effectively inhibited. Particularly, the system could inhibit the dopa oxidase activity even at a very low concentration, as compared with kojic acid, which is a known skin-lightening component. Furthermore, as described above, the dopa oxidase activity is used as an index for the enzyme activity of tyrosinases which is involved in melanin biosynthesis. Therefore, it is understood that toosendanin can suppress melanin production by inhibiting the dopa oxidase activity, and is useful as a skin-lightening component.

### Prescription Example 1

A lotion, an emulsion, a serum, a cream and a facial pack having the compositions shown below were respectively prepared by conventional methods, using the extracts obtained in Preparation Examples 1 to 4 as active ingredients.

| | |
|---|---|
| (Component) | (Content: mass %) |

1. Preparation of lotion

| | |
|---|---|
| 1,3-Butylene glycol | 8.0 |
| Glycerol | 5.0 |
| Ethanol | 3.0 |
| Extract of Melia toosendan Sieb. et Zucc. | 3.0 |
| Chamomile extract | 3.0 |
| Bellflower extract | 1.0 |
| Clove extract | 1.0 |
| Xanthane gum | 0.1 |
| Hyaluronic acid | 0.1 |
| Disodium hydrogen phosphate | 0.1 |
| Sodium dihydrogen phosphate | 0.1 |
| Glucoside 2-ascorbate | 2.0 |
| Purified water | Balance |
| Perfume | Moderate amounts |
| Preservative | Moderate amounts |

2. Preparation of lotion

| | |
|---|---|
| Extract of Amomum tsao-ka Crevost et Lemaire | 3.0 |
| 1,3-Butylene glycol | 5.0 |
| Glycerol | 3.0 |
| Propylene glycol | 3.0 |
| Ethanol | 3.0 |
| Chamomile extract | 2.0 |
| Xanthane gum | 0.1 |
| Hyaluronic acid | 0.1 |
| Disodium hydrogen phosphate | 0.1 |
| Sodium dihydrogen phosphate | 0.1 |
| Purified water | Balance |
| Perfume | Moderate amounts |
| Preservative | Moderate amounts |

3. Preparation of emulsion

| | |
|---|---|
| Extract of Senecio gracilis | 5.0 |
| Chamomile extract | 1.0 |
| Bellflower extract | 1.0 |
| Althea extract | 2.0 |
| Squalane | 3.0 |
| Oil of olive | 3.0 |
| Glycerol | 5.0 |
| Polyoxyethylene hydrogenated castor oil | 1.0 |
| (Number of added moles of ethylene oxide: 40) | |
| Carboxyvinyl polymer | 0.2 |
| Potassium hydroxide | 0.1 |
| Xanthane gum | 0.2 |
| Disodium edetate | 0.02 |
| Purified water | Balance |
| Preservative | Moderate amounts |

4. Preparation of serum

| | |
|---|---|
| Extract of Veratrum nigrum L. | 5.0 |
| Chamomile extract | 1.0 |
| Bellflower extract | 1.0 |
| Clove extract | 1.0 |
| Carboxyvinyl polymer | 0.2 |
| Acrylate•methacrylate alkyl copolymer | 0.2 |
| Potassium hydroxide | 0.2 |
| Xanthane gum | 0.1 |
| Hyaluronic acid | 0.2 |
| Sodium citrate | 0.15 |
| Citric acid | 0.03 |
| Glycerol | 10.0 |
| 1,3-Butylene glycol | 5.0 |
| Disodium edetate | 0.05 |
| Purified water | Balance |
| Preservative | Moderate amounts |
| Perfume | Moderate amounts |

5. Preparation of serum

| | |
|---|---|
| Extract of Melia toosendan Sieb. et Zucc. | 3.0 |
| Chamomile extract | 1.0 |
| Bellflower extract | 1.0 |
| Clove extract | 1.0 |
| Xanthane gum | 0.2 |
| Carboxymethyl cellulose | 0.2 |
| Carboxyvinyl polymer | 0.2 |
| Potassium hydroxide | 0.1 |
| Citric acid | 0.03 |
| Sodium citrate | 0.15 |
| Glycerol | 5.0 |
| Propylene glycol | 3.0 |
| Polyethyleneglycol (number average | 1.0 |
| molecular weight 1500) | |
| Polyethylene glycol monostearate | 0.5 |
| Purified water | Balance |
| Preservative | Moderate amounts |
| Perfume | Moderate amounts |

6. Preparation of cream

| | |
|---|---|
| Extract of Amomum tsao-ka Crevost et Lemaire | 3.0 |
| Chamomile extract | 2.0 |
| Bellflower extract | 2.0 |
| Clove extract | 2.0 |
| Methylpolysiloxane | 3.0 |
| Squalane | 2.0 |
| Neopentyl glycol dicaprate | 3.0 |
| Stearyl alcohol | 1.5 |
| Cetanol | 1.0 |
| Polyoxyethylene hydrogenated castor oil | 0.5 |
| (Number of added moles of ethylene oxide: 60) | |
| Acrylate•methacrylate alkyl copolymer | 0.3 |
| Potassium hydroxide | 0.15 |
| Xanthane gum | 0.1 |
| Disodium edetate | 0.05 |
| Purified water | Balance |
| Preservative | Moderate amounts |
| Perfume | Moderate amounts |

7. Preparation of cream

| | |
|---|---|
| Extract of Senecio gracilis | 2.0 |
| Chamomile extract | 3.0 |
| Bellflower extract | 3.0 |
| Polyoxyethylene alkyl ether-modified silicone | 3.0 |
| Methylpolysiloxane | 5.0 |
| Decamethylcyclopentasiloxane | 10 |
| Squalane | 5.0 |
| Magnesium sulfate | 0.5 |
| Glycerol | 5.0 |
| 1,3-Butylene glycol | 5.0 |
| Purified water | Balance |
| Preservative | Moderate amounts |
| Perfume | Moderate amounts |

8. Preparation of facial pack

| | |
|---|---|
| Extract of Veratrum nigrum L. | 3.0 |
| Chamomile extract | 2.0 |
| Bellflower extract | 1.0 |
| Dipropylene glycol | 3.0 |
| Polyethylene glycol (number average | 2.0 |
| molecular weight 1500) | |
| 1,3-Butylene glycol | 5.0 |
| Glycerol | 5.0 |
| Sodium citrate | 0.5 |
| Polyvinyl alcohol | 10 |
| Lactic acid | 0.3 |
| Polyoxyethylene decyl tetradecyl ether | 0.5 |
| Purified water | Balance |
| Preservative | Moderate amounts |
| Perfume | Moderate amounts |

### Prescription Example 2

A lotion, an emulsion, a serum, a cream and a facial pack having the compositions shown below were respectively prepared by conventional methods, using toosendanin as active ingredients.

| | |
|---|---|
| (Component) | (Content: mass %) |

1. Preparation of lotion

| | |
|---|---|
| Toosendanin | 0.1 |
| 1,3-Butylene glycol | 8.0 |
| Glycerol | 5.0 |
| Ethanol | 3.0 |
| Chamomile extract | 3.0 |
| Bellflower extract | 1.0 |
| Clove extract | 1.0 |
| Xanthane gum | 0.1 |
| Hyaluronic acid | 0.1 |
| Disodium hydrogen phosphate | 0.1 |
| Sodium dihydrogen phosphate | 0.1 |
| Glucoside 2-ascorbate | 2.0 |
| Purified water | Balance |
| Perfume | Moderate amounts |
| Preservative | Moderate amounts |

2. Preparation of emulsion

| | |
|---|---|
| Toosendanin | 0.5 |
| Chamomile extract | 1.0 |
| Bellflower extract | 1.0 |
| Althea extract | 2.0 |
| Squalane | 3.0 |
| Oil of olive | 3.0 |
| Glycerol | 5.0 |
| Polyoxyethylene hydrogenated castor oil | 1.0 |
| (Number of added moles of ethylene oxide: 40) | |
| Carboxyvinyl polymer | 0.2 |
| Potassium hydroxide | 0.1 |
| Xanthane gum | 0.2 |
| Disodium edetate | 0.02 |
| Purified water | Balance |
| Preservative | Moderate amounts |

3. Preparation of serum

| | |
|---|---|
| Toosendanin | 0.5 |
| Chamomile extract | 1.0 |
| Bellflower extract | 1.0 |
| Clove extract | 1.0 |
| Carboxyvinyl polymer | 0.2 |
| Acrylate•methacrylate alkyl copolymer | 0.2 |
| Potassium hydroxide | 0.2 |
| Xanthane gum | 0.1 |
| Hyaluronic acid | 0.2 |
| Citric citrate | 0.15 |
| Citric acid | 0.03 |
| Glycerol | 10.0 |
| 1,3-Butylene glycol | 5.0 |
| Disodium edetate | 0.05 |
| Purified water | Balance |
| Preservative | Moderate amounts |
| Perfume | Moderate amounts |

4. Preparation of cream

| | |
|---|---|
| Toosendanin | 1.0 |
| Chamomile extract | 2.0 |
| Bellflower extract | 2.0 |
| Clove extract | 2.0 |
| Methylpolysiloxane | 3.0 |
| Squalane | 2.0 |
| Neopentyl glycol dicaprate | 3.0 |
| Stearyl alcohol | 1.5 |
| Cetanol | 1.0 |
| Polyoxyethylene hydrogenated castor oil | 0.5 |
| (Number of added moles of ethylene oxide: 60) | |
| Acrylate•methacrylate alkyl copolymer | 0.3 |
| Potassium hydroxide | 0.15 |
| Xanthane gum | 0.1 |
| Disodium edetate | 0.05 |
| Purified water | Balance |
| Preservative | Moderate amounts |
| Perfume | Moderate amounts |

5. Preparation of facial pack

| | |
|---|---|
| Toosendanin | 1.0 |
| Chamomile extract | 2.0 |
| Bellflower extract | 1.0 |
| Dipropylene glycol | 3.0 |
| Polyethylene glycol (number average | 2.0 |
| molecular weight 1500) | |
| 1,3-Butylene glycol | 5.0 |
| Glycerol | 5.0 |
| Sodium citrate | 0.5 |
| Polyvinyl alcohol | 10 |
| lactic acid | 0.3 |
| Polyoxyethylene decyl tetradecyl ether | 0.5 |
| Purified water | Balance |
| Preservative | Moderate amounts |
| Perfume | Moderate amounts |

Having described our invention as related to the present embodiments, it is our intention that the present invention not be limited by any of the details of the description, unless otherwise specified, but rather be construed broadly within its spirit and scope as set out in the accompanying claims.

This non-provisional application claims priority under 35 U.S.C. §119 (a) on Patent Application No. 2009-263588 filed in Japan on Nov. 19, 2009, which is entirely herein incorporated by reference.

What is claimed is:

1. A dopa oxidase inhibitor, comprising an extract of at least one plant selected from the group consisting of Melia toosendan Sieb. et Zucc., Amomum tsao-ka Crevost et Lemaire, Senecio gracilis and Veratrum nigrum L. as an active ingredient.

2. A skin-lightening agent, comprising an extract of at least one plant selected from the group consisting of Melia toosendan Sieb. et Zucc., Amomum tsao-ka Crevost et Lemaire, Senecio gracilis and Veratrum nigrum L. as an active ingredient.

3. An external preparation for skin, comprising an extract of at least one plant selected from the group consisting of Melia toosendan Sieb. et Zucc., Amomum tsao-ka Crevost et Lemaire, Senecio gracilis and Veratrum nigrum L. as an active ingredient.

4. A dopa oxidase inhibitor, containing the compound represented by the following Formula (1) as an active ingredient: 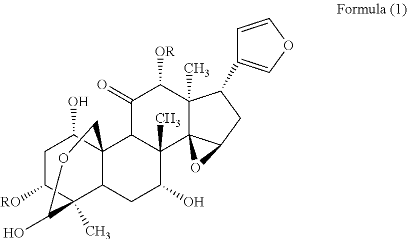
wherein R represents an acyl group having 1 to 5 carbon atom(s).

5. A skin-lightening agent, comprising the compound represented by the following Formula (1) as an active ingredient: 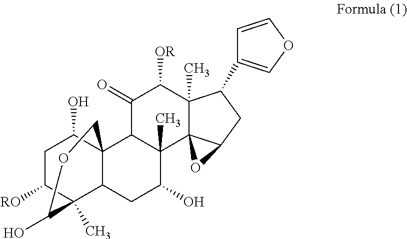
wherein R represents an acyl group having 1 to 5 carbon atom(s).

6. A method for inhibiting a dopa oxidase activity, comprising applying an extract of at least one plant selected from the group consisting of Melia toosendan Sieb. et Zucc., Amomum tsao-ka Crevost et Lemaire, Senecio gracilis and Veratrum nigrum L. to a skin.

7. A method for lightening a skin, comprising applying an extract of at least one plant selected from the group consisting of Melia toosendan Sieb. et Zucc., Amomum tsao-ka Crevost et Lemaire, Senecio gracilis and Veratrum nigrum L. to a skin.

8. A method for inhibiting a dopa oxidase activity, comprising applying the compound represented by the following Formula (1) to a skin: 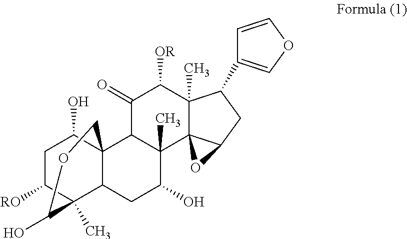
wherein R represents an acyl group having 1 to 5 carbon atom(s).

9. A method for lightening a skin, comprising applying the compound represented by the following Formula (1) to a skin: 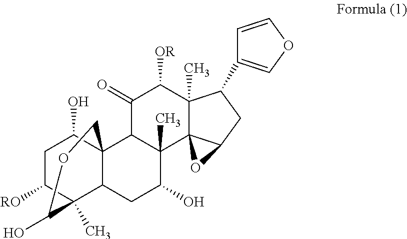
wherein R represents an acyl group having 1 to 5 carbon atom(s).
